(19) United States
(12) Patent Application Publication
(10) Pub. No.: US 2007/0049819 A1
(43) Pub. Date: Mar. 1, 2007

(54) Bone fixed locater and optical navigation system

(76) Inventors: Jan Stifter, Unterehrendingen (CH); Thomas Hauri, Staffelbach (CH)

Correspondence Address: KNOBBE MARTENS OLSON & BEAR LLP, 2040 MAIN STREET, FOURTEENTH FLOOR, IRVINE, CA, 92614, US

(21) Appl. No.: 10/573,144

(22) PCT Filed: Sep. 15, 2004
(86) PCT No.: PCT/EP04/10347

§ 371 Date: Oct. 13, 2006

(30) Foreign Application Priority Data
Sep. 22, 2003 (DE) 10343826.2

(51) Int. Cl. A61B 5/05

(52) U.S. Cl. 600/426; 600/407

(57) ABSTRACT

Bone-fixed locator (3, 5) as reference of a navigation system (1) for determining the spatial position and location of body parts of a mammal, having a recording device, especially a stereo-camera arrangement (9), for locating the position of locators on the basis of signals provided by target markers on the locators and having a control and evaluation device (11) connected to the recording device, there being fewer than three target markers (3c, 3d, 5c, 5d) provided on a body (3a, 5a) for giving a signal to the recording device and an engagement portion (3b, 5b) configured for engagement in a bone of the mammal.

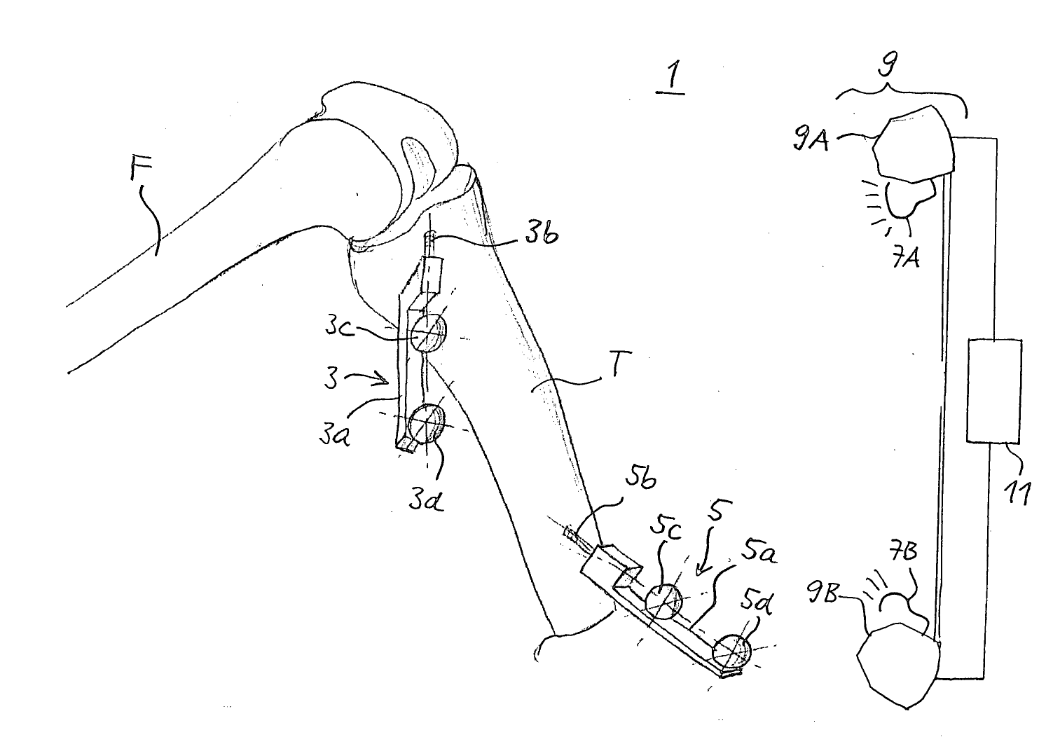

The invention relates to a bone-fixed locator of a so-called navigation system for medical use as well as to such a system that comprises locators of that kind.

Methods and apparatus for pre-operative or intra-operative determination of the position or alignment of limbs of a mammal, especially a human being, and/or of surgical instruments and/or of endoprosthetic parts intended for insertion into the body have been known for some years and are increasingly being used clinically.

For example, a method and an apparatus for determining the centre of rotation of joints of the human body, especially the hip joint or knee joint, by means of an optical detection system are known from FR 2 785 517. WO 95/00075 describes a method and an apparatus for detecting the position of functional structures of the lower leg during knee surgery using a navigation system of the kind in question. A similar method is also known from WO 99/23956 which teaches the substantially simultaneous use of bone-fixed locators (referred to herein as reference bodies) and manually manipulatable locators, also referred to as hand-guided scanners. DE 197 09 960 A1 describes a method and an apparatus for the pre-operative determination of position data of endoprosthetic parts of a middle joint of the human body relative to the adjacent bones.

The bone-fixed locators used in those systems (marker elements, reference bodies or the like) usually have three or more radiation transmitters (IR-LED) or radiation reflectors (IR reflectors), which allows independent detection of their spatial position. For that purpose, basically three transmitter or reflector elements are sufficient, whereas 4 or more is redundant from the physical standpoint, but allows additional testing steps or information relating to the conclusiveness of the position data obtained.

In many cases, such locators are approximately T-shaped or Y-shaped, one of the transmitter or reflector elements often being arranged in an elevated position relative to a plane in which the other three are located, but there are also locators having a substantially rectangular or rhombohedral body with an attached rod-shaped holder, as well as locators having only three transmitter or reflector elements on a substantially linear body.

A common feature of all those locators is that—taking into consideration the distance between the transmitter and reflector elements that is necessary for reliable detection by the optical detection device—they have relatively large dimensions and, on account of their necessary rigidity, are also relatively heavy. For fixing to the bone there are therefore usually used elaborate clamping or screwing devices of relatively large volume which require the exposure of a correspondingly large area of the bone region, which goes against general medical concern that any necessary interventions should be minimally invasive. Simpler fixing means, such as the self-tapping thread screws described in DE 197 09 960 A1 to be screwed directly into the bone portion, have not become established in view of the relatively high loads of conventional locators.

The invention is therefore based on the problem of providing a smaller, lighter locator of the kind mentioned in the preamble that can be attached to the bone in a minimally invasive way. A further objective is to provide a medical navigation system matched to such improved locators.

That problem is solved in its first aspect by a locator having the features of claim 1 and in its second aspect by a navigation system having the features of claim 6.

The invention includes the fundamental concept of doing without the independent position locatability, i.e. the possibility of determining a complete set of position data of an individual locator with regard to the specific conditions of the medical use. Those specifics are that the body part the spatial position or orientation of which is to be determined can as such provide a rigid connection between two or more locator elements which are in turn rigidly connected to that body part (bone, joint part etc.). The invention is therefore based to a certain extent on the concept of using functionally incomplete, reduced locators which allow a complete set of position data to be acquired only when rigidly connected to one another by way of the bone.

The invention now makes it possible for locators of substantially smaller and lighter structure to be fixed using a fixing that is far less invasive than conventional locators, because the effective forces are substantially reduced. In addition, there is advantageously a significant reduction in the undesirable effect on ligaments in the operating region and/or irritation to adjacent soft tissue. It is clear that the reduced size and the reduced weight as well as the simplified fixing mechanism also give rise to greater flexibility in respect of the use and specific placement of the locators according to the invention.

A further advantage over known navigation principles is that the measurement basis between the reference points or target markers (transmitter or reflector elements) that are to be related to one another is broadened in comparison with completely independent acquisition of position data from the measurement signals of individual locators. In the case of the latter, the extent of the measurement basis in respect of its total size and its weight is naturally very limited, whereas in the proposed solution the placement of the locators which together deliver a set of position data can be selected within wide limits and therefore, if necessary, a considerably enlarged measurement basis can be implemented. It should also be noted here that small errors arising during system operation, for example unintentional displacements of an individual locator, have a less marked effect on the total result of the position determination when the measurement basis is enlarged.

Finally, it should be pointed out that the simplified fixing allows complete rotation of the locator about its axis at any time, which enables the optical detection situation to be optimised with very little effort. Although it is also possible to implement rotatable bone-fixed locators in a conventional structure, it would require additional joints or pivot axes on the fixing device which would accordingly be made even bigger and heavier. Moreover, in the case of conventional navigation systems, on account of the different measurement principle, rotation of the locators is allowed only prior to the start of the detection procedure but is forbidden afterwards.

For implementing this advantageous rotatability, in a locator configuration having two target markers the pivot axis lies especially in a line connecting those target markers. In the case of spherical target markers, which are commonly used, that line is defined between the centre points of the spheres, but in the case of planar target markers it may also lie outside the centre points of the respective planes.

In a preferred configuration, the locator comprises two reflector or transmitter elements provided on a substantially linear or L-shaped body. A locator having a single reflector or transmitter element also lies within the scope of the invention, but its use in a medical navigation system requires the bone-fixed fixing and evaluation-side combination of signals of at least three locators and is less advantageous than the configuration having two markers also in terms of error reliability.

In a further preferred configuration, the engagement portion is in the form of a self-tapping thread—which is more preferably also self-drilling. Using such a screw, the locator according to the invention can be reliably fixed to the bone or bone portion the position of which is to be determined using a small number of simple manipulations and with little invasiveness. Positional displacements in the longitudinal direction are virtually ruled out, as is unintentional loosening. In a locator configuration having two target markers, the thread axis lies preferably in the (above-mentioned) line connecting them.

In principle, the proposed locator can also be configured with a fixing based solely on frictional force in accordance with the nail principle. In such a configuration, the locator is likewise easy to manipulate—albeit using an additional tool (hammer)—but the stability of such a fixing does not approach that of a thread.

In an especially simple configuration, the longitudinal axis of the fixing element extends substantially in the axis of the markers, but configurations having a fixing thread that is inclined relative to the locator body are also possible in principle. In such configurations, however, rotation of the locator about its own axis would be a geometrically more complex operation, and unintentional small rotations about the longitudinal axis of the thread would become noticeable in the position of the markers (reflector or transmitter elements).

In a further preferred configuration of the proposed locator, the reflector elements are in the form of retro-reflecting spheres, that is to say the locator is of the passive type. On account of its lesser complexity and greater flexibility of use arising from not having its own power supply, such a passive configuration is especially advantageously integrated into the concept on which the invention is based.

For that purpose, especially, there is implemented in the control and evaluation device (11) an evaluation program for the associated evaluation of signals provided by target markers (3c, 3d, 5c, 5d) on the two or more locators (3, 5) so that the signals of at most two target markers on one and the same locator are entered in the position determination. Even when, therefore, locators having three or more target markers are to be used in a navigation system of the kind according to the invention, that configuration of the control and evaluation device ensures that the position determination is carried out on the basis of the data of a plurality of locators rigidly connected by way of the bone and not on the basis of the signals of an individual locator. In that respect, locators having, for example, three or more target markers of which, however, only two are used in the sense of the invention would also be regarded as locators lying within the scope of the invention.

Advantages and useful features of the invention will otherwise be found in the dependent claims as well as in the following description of a preferred embodiment with reference to the single FIGURE.

The FIGURE shows a simplified diagrammatic view of an optical navigation system 1 for medical use, the important components of which are two bone-fixed locators 3 and 5 of the kind according to the invention, IR illumination sources 7A and 7B, a stereo-camera arrangement 9 having two IR cameras 9a and 9b and an evaluation unit 11 connected thereto. In respect of the illumination unit 7 and the stereo-camera arrangement 9, the system has no special features in comparison with known navigation systems. In the evaluation unit 11, however, a special evaluation program is implemented which allows combined evaluation of the light signals coming from the two locators 3 and 5 to create a set of position data.

The locators 3 and 5 are identically constructed, each having an L-shaped body 3a and 5a, a self-cutting thread 3b and 5b mounted thereon and two retro-reflecting spheres 3c, 3d and 5c, 5d, respectively, on the elongate portion of the body. They are screwed into a tibia T of a patient, spaced apart from one another, and define the reference of the tibia T. Screwing-in is effected without any appreciable prior exposure of the fixing region and is largely free of soft-tissue irritation and impairment of the ligaments in the knee joint or foot joint region.

The invention is not limited to that example, but can also be realized in a plurality of modifications that lie within the scope of technical action. In particular, modifications in respect of the shape of the locator body and the nature and arrangement of the associated fixing devices (as described above) are possible, as are also, however, modifications in respect of the number, shape and arrangement of the marker elements on the locators.

1-10. (canceled)

11. A bone-fixed locator for use with a navigation system for determining the spatial position and location of a body part of a mammal based on signals from the locator, the navigation system having a recording device connected to a control an evaluation device thereof, the bone-fixed locator comprising:
a body with fewer than three target markers, the target markers configured to communicate a signal to a recording device of a navigation system; and
an engagement portion attached to the body, the engagement portion configured for engagement with a bone of a mammal.

12. The locator of claim 11, wherein the engagement portion comprises a self-drilling, self-tapping thread.

13. The locator of claim 11, wherein the fewer than three target markers comprises two target markers that extend along a pivot axis of the body.

14. The locator of claim 13, wherein the engagement portion extends along the pivot axis, the locator being pivotable about the pivot axis.

15. The locator of claim 11, wherein the target markers comprise two reflector or transmitter elements provided on the body, the body selected from a group consisting of a substantially linear body and an L-shaped body, the reflector or transmitter elements configured to communicate a signal to an optical recording device.

16. The locator of claim 15, wherein the optical recording device comprises a stereo-camera arrangement.

17. The locator of claim 15, wherein the reflector or transmitter elements comprise retro-reflecting spheres.

18. A navigation system for determining the spatial position and location of a body part of a mammal, comprising:
a recording device;
a control and evaluation device connected to the recording device; and
at least two locators rigidly fastened to a bone, the locators operably connected to one another via the bone, each locator comprising a body and fewer than three target markers mounted thereon,
wherein the recording device is configured to receive signals from the target markers, and wherein the control and evaluation device is configured to evaluate said signals to establish a bone-fixed co-ordinate system.

19. The navigation system of claim 18, wherein the control and evaluation device comprises an evaluation program configured to evaluate the signals provided by the target markers so that the signals of at most two target markers on each locator are used to determine the position of the body part of the mammal.

20. The navigation system of claim 18, wherein the at least two locators comprises two locators.

21. The navigation system of claim 18, wherein the recording device comprises a stereo-camera arrangement.

22. A method for determining the spatial position and location of body parts of a mammal, comprising:
fastening at least two locators to a bone, the locators operably connected to one another via the bone, each locator defining a pivot axis extending between fewer than three target markers on the locator and an engagement portion of the locator, the locator pivotable about the pivot axis;
recording signals communicated by each target marker; and
evaluating said signals to determine the spatial position and location of a body part.

23. The method of claim 22, wherein fastening the at least two locators to the bone includes rigidly fastening the locators to the bone in a minimally-invasive manner without appreciable prior exposure of a fixing region on the bone and so as to inhibit soft-tissue irritation and impairment of the ligaments in a corresponding joint of the bone.

24. The method of claim 22, wherein fastening the at least two locators to a bone comprises screwing the locator into the bone.

25. The method of clam 22, wherein evaluating the signals includes running an evaluation program implemented in a control and evaluation device.

26. The method according to claim 22, wherein evaluating the signals includes pivoting at least one of the locators about the pivot axis following a start of the evaluation process.
